(19) United States
(12) Patent Application Publication
(10) Pub. No.: US 2026/0053376 A1
(43) Pub. Date: Feb. 26, 2026

(54) INFORMATION PROCESSING APPARATUS, INFORMATION PROCESSING METHOD, AND PROGRAM

(71) Applicant: SONY GROUP CORPORATION, TOKYO (JP)

(72) Inventors: KIMINOBU NISHIMURA, TOKYO (JP); YOHEI KAWAMOTO, TOKYO (JP); ARINOBU UEDA, TOKYO (JP); SAI KARASAWA, TOKYO (JP)

(21) Appl. No.: 19/103,212

(22) PCT Filed: Jun. 29, 2023
(86) PCT No.: PCT/JP2023/024242

§ 371 Date: Feb. 11, 2025

(30) Foreign Application Priority Data
Aug. 18, 2022 (JP) 2022-130672

(51) Int. Cl. A61B 5/021; A61B 5/00; A61B 5/024

(52) U.S. Cl. CPC A61B 5/02125; A61B 5/02416; A61B 5/7278

(57) ABSTRACT

To derive a pulse wave propagation speed with higher accuracy. An information processing apparatus includes a calculation unit configured to derive a pulse wave propagation speed of a subject by performing signal processing together on pulse wave signals sensed at three or more different positions on a body surface of the subject.

### TECHNICAL FIELD

The present disclosure relates to an information processing apparatus, an information processing method, and a program.

### BACKGROUND ART

In recent years, in order to prevent a lifestyle disease at a stage before becoming a disease, it has been studied to monitor a health condition of a subject in daily life. Specifically, it has been studied to measure a blood pressure of the subject in each life scene.

Generally, the blood pressure is measured by a sphygmomanometer in which an arm cuff (so-called cuff) is wound around the arm of the subject. However, in such a measurement method, the arm of the subject is tightened with the cuff, and thus, the burden on the subject is large. Thus, a technology for measuring the blood pressure of the subject with a low load without using a cuff has been desired.

For example, Patent Document 1 below discloses a technology for calculating a blood pressure on the basis of a pulse wave acquired from a plurality of regions on the body surface of a subject. In the technology disclosed in Patent Document 1, at least two regions are selected according to signal quality of the pulse wave from among a plurality of regions where the pulse wave is acquired, and the blood pressure is calculated on the basis of pulse wave propagation between the selected regions.

### CITATION LIST

### Patent Document

Patent Document 1: WO 2019/116996 A

### SUMMARY OF THE INVENTION

### Problems to be Solved by the Invention

However, in the technology disclosed in Patent document 1 described above, in a case where a distance between the two regions where the pulse wave is acquired is short, as a result of a period required for propagation of the pulse wave being shortened, accuracy of blood pressure estimation may significantly degrade.

The present disclosure therefore proposes a new and improved information processing apparatus, information processing method, and program capable of deriving a pulse wave propagation speed with higher accuracy.

### Solutions to Problems

According to the present disclosure, there is provided an information processing apparatus including a calculation unit configured to derive a pulse wave propagation speed of a subject by performing signal processing together on pulse wave signals sensed at three or more different positions on a body surface of the subject.

Furthermore, according to the present disclosure, there is provided an information processing method including deriving, by an arithmetic processing device, a pulse wave propagation speed of a subject by performing signal processing together on pulse wave signals sensed at three or more different positions on a body surface of the subject.

Furthermore, according to the present disclosure, there is provided a program for causing a computer to function as a calculation unit configured to derive a pulse wave propagation speed of a subject by performing signal processing together on pulse wave signals sensed at three or more different positions on a body surface of the subject.

### BRIEF DESCRIPTION OF DRAWINGS

FIG. 1 is a block diagram illustrating a functional configuration of an information processing apparatus according to a first embodiment of the present disclosure.

FIG. 2 is an explanatory diagram for explaining a specific example of processing by the information processing apparatus according to the first embodiment.

FIG. 3 is a flowchart indicating flow of operation of the information processing apparatus according to the first embodiment.

FIG. 4 is a schematic view illustrating an appearance of a measurement device according to a first example of the first embodiment.

FIG. 5 is a schematic view illustrating an extracted measurement unit of the measurement device according to the first example of the first embodiment.

FIG. 6 is a schematic view illustrating an appearance of a measurement device according to a second example of the first embodiment.

FIG. 7 is a schematic view illustrating an appearance of a measurement device according to a third example of the first embodiment.

FIG. 8 is a schematic view illustrating an appearance of a measurement device according to a fourth example of the first embodiment.

FIG. 9 is a schematic view illustrating an appearance of a measurement device according to a fifth example of the first embodiment.

FIG. 10 is a schematic view illustrating an appearance of a measurement device according to a sixth example of the first embodiment.

FIG. 11 is a block diagram illustrating a functional configuration of an information processing apparatus according to a second embodiment of the present disclosure.

FIG. 12 is an explanatory diagram for explaining a specific example of processing by the information processing apparatus according to the second embodiment.

FIG. 13 is a flowchart indicating flow of operation of the information processing apparatus according to the second embodiment.

FIG. 14 is a schematic view illustrating an appearance of a measurement device according to a first example of the second embodiment.

FIG. 15 is a schematic view illustrating an appearance of a measurement device according to a second example of the second embodiment.

FIG. 16 is a schematic view illustrating an appearance of a measurement device according to a third example of the second embodiment.

FIG. 17 is a schematic view illustrating an appearance of a measurement device according to a fourth example of the second embodiment.

FIG. 18 is a schematic view illustrating an appearance of a measurement device according to a fifth example of the second embodiment.

FIG. 19 is a block diagram illustrating a functional configuration of an information processing apparatus according to a third embodiment of the present disclosure.

FIG. 20 is an explanatory diagram for explaining a specific example of processing by the information processing apparatus according to the third embodiment.

FIG. 21 is an explanatory diagram for explaining a specific example of the processing by the information processing apparatus according to the third embodiment.

FIG. 22 is a flowchart indicating flow of operation of the information processing apparatus according to the third embodiment.

FIG. 23 is a schematic view illustrating a correction mechanism that corrects a sensing position of light emitted from a sensor in order to improve signal quality from the sensor.

FIG. 24 is a schematic view illustrating a correction mechanism that corrects a sensing position of light emitted from a sensor in order to improve signal quality from the sensor.

FIG. 25 is a schematic view illustrating an appearance of a measurement device according to a first example of the third embodiment.

FIG. 26 is a schematic view illustrating an appearance of a measurement device according to a second example of the third embodiment.

FIG. 27 is a schematic view illustrating an appearance of a measurement device according to a third example of the third embodiment.

FIG. 28 is a schematic view illustrating an appearance of a measurement device according to a fourth example of the third embodiment.

### MODE FOR CARRYING OUT THE INVENTION

Hereinafter, preferred embodiments of the present disclosure will be described in detail with reference to the accompanying drawings. Note that, in the present specification and drawings, components having substantially the same functional configuration are denoted by the same reference signs, and redundant description is omitted.

Note that the description will be given in the following order.

1. First Embodiment

1.1. Configuration Example of Information Processing Apparatus

1.2. Operation Example of Information Processing Apparatus

1.3. Examples of Measurement Device

2. Second Embodiment

2.1. Configuration Example of Information Processing Apparatus

2.2. Operation Example of Information Processing Apparatus

2.3 Examples of Measurement Device

3. Third Embodiment

3.1. Configuration Example of Information Processing Apparatus

3.2. Operation Example of Information Processing Apparatus

3.3. Modifications

3.4. Example of Measurement Device

### 1. FIRST EMBODIMENT

### 1.1. Configuration Example of Information Processing Apparatus

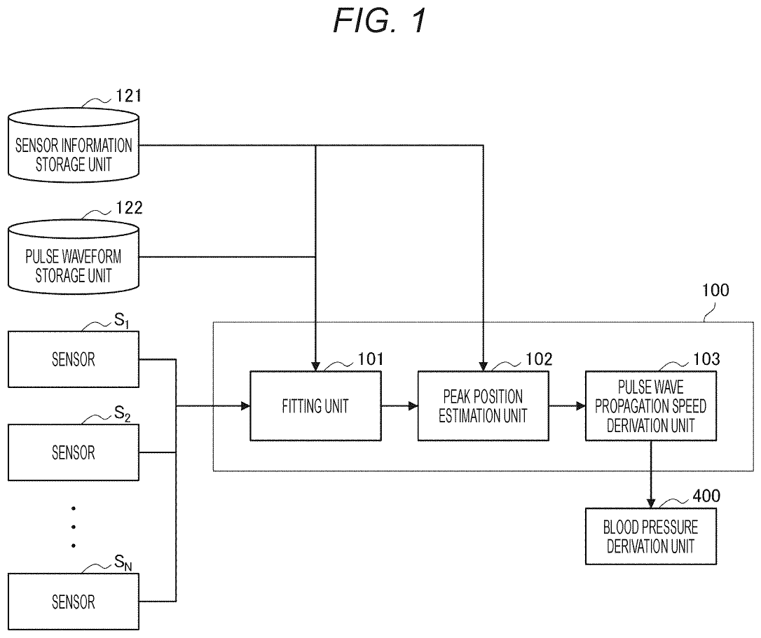

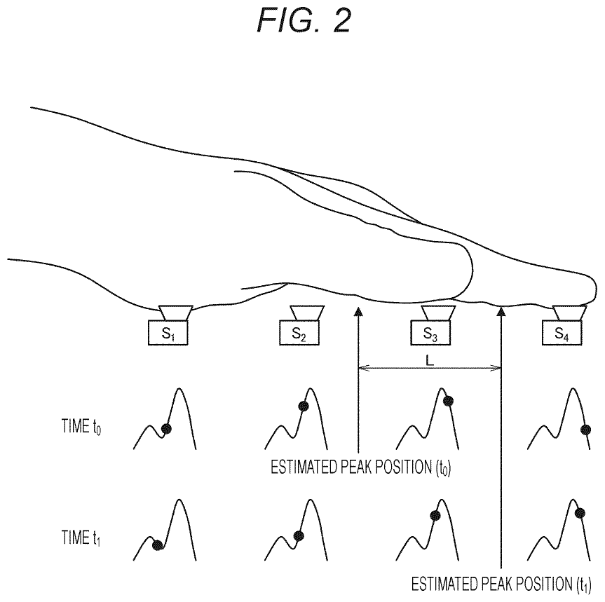

First, a configuration of an information processing apparatus according to a first embodiment of the present disclosure will be described with reference to FIGS. 1 and 2. FIG. 1 is a block diagram illustrating a functional configuration of an information processing apparatus 100 according to the present embodiment. FIG. 2 is an explanatory diagram for describing a specific example of processing by the information processing apparatus 100.

As illustrated in FIG. 1, the information processing apparatus 100 includes a fitting unit 101, a peak position estimation unit 102, and a pulse wave propagation speed derivation unit 103. The information processing apparatus 100 can estimate a peak position of a pulse wave signal by fitting each of the pulse wave signals sensed by a plurality of sensors S₁ to S_{N} to a predetermined pulse waveform and derive a pulse wave propagation speed on the basis of a difference in the peak position of the pulse wave signal at different times.

The fitting unit 101 fits a predetermined pulse waveform to each of the pulse wave signals sensed by the plurality of sensors S₁ to S_{N}. Specifically, the fitting unit 101 fits a predetermined pulse waveform to the pulse wave signals sensed at the same time by the plurality of sensors S₁ to S_{N} using a least squares method. For example, the fitting unit 101 may perform fitting by expanding and contracting a predetermined pulse waveform such that the predetermined pulse waveform passes through respective points obtained by plotting distances between the plurality of sensors S₁ to S_{N} on a horizontal axis and sensing values of the plurality of sensors S₁ to S_{N} on the vertical axis. Note that in the fitting, a limit such as an upper limit or a lower limit may be provided for expansion and contraction of the predetermined pulse waveform in a time direction and an amplitude direction.

The plurality of sensors S₁ to S_{N} is a sensor that senses a pulse wave on the body surface of the subject.

The plurality of sensors S₁ to S_{N} may be a sensor that optically detects a pulse wave without winding a cuff around the arm, or the like, of the subject. For example, the plurality of sensors S₁ to S_{N} may be a sensor using laser Doppler flowmetry (LDF) or a sensor using photoplethysmography (PPG). The plurality of sensors S₁ to S_{N} can sense a pulse wave propagating through the same arterial path at different positions by sensing a pulse wave at at least three or more different positions on the same arterial path. In order to perform fitting with high accuracy, the number of sensors S₁ to S_{N} is desirably three or more. An upper limit of the number of the sensors S₁ to S_{N} is not particularly limited, but may be, for example, 10 from the viewpoint of cost and installation location.

The predetermined pulse waveform is an ideal pulse waveform of the subject. As an example, the predetermined pulse waveform may be a representative pulse waveform of a human set on the basis of an attribute such as age, body shape, or sex of the subject. As another example, the predetermined pulse waveform may be a waveform derived by measuring a pulse wave of the subject for a certain period or more and averaging waveforms of the measured pulse wave. The predetermined pulse waveform is stored in advance in a pulse waveform storage unit 122. The pulse waveform storage unit 122 may include a magnetic storage device such as a hard disk drive (HDD), a semiconductor storage device, an optical storage device, a magneto-optical storage device, or the like.

For example, as illustrated in FIG. 2, in a case where four sensors S₁ to S₄ are provided between the wrist and the fingertip of the hand of the subject, the four sensors S₁ to S₄ sense signals at different positions of the pulse waveform along with propagation of the pulse wave through the same arterial path. In such a case, the fitting unit 101 fits a predetermined pulse waveform to a graph obtained by plotting pulse wave signals sensed by the four sensors S₁ to S₄ with a positional relationship between the four sensors S₁ to S₄ as a horizontal axis. Thus, the fitting unit 101 can complement and estimate a pulse waveform between the sensors S₁ to S₄ with the fitted pulse waveform.

The peak position estimation unit 102 estimates a peak position of the sensed pulse wave signal on the basis of the fitted pulse waveform. Specifically, the peak position estimation unit 102 may estimate the peak position of the fitted pulse waveform as the peak position of the sensed pulse wave signal. Note that the peak position of the pulse wave signal may be expressed by a positional relationship with the plurality of sensors S₁ to S_{N}. For example, the peak position of the pulse wave signal may be expressed by identification information of the sensors S₁ to S_{N} before and after the peak position and a ratio of distances to the sensors S₁ to S_{N} before and after the peak position.

The positional relationship between the plurality of sensors S₁ to S_{N} may be expressed by a distance between each other on the arterial path. For example, in a case where the plurality of sensors S₁ to S_{N} is provided on a linear arterial path, the positional relationship between the plurality of sensors S₁ to S_{N} may be expressed by a linear distance therebetween. In addition, in a case where the plurality of sensors S₁ to S_{N} is provided on a curved arterial path, the positional relationship between the plurality of sensors S₁ to S_{N} may be expressed by a distance along the arterial path.

Information regarding the positions of the plurality of sensors S₁ to S_{N} is stored in advance in the sensor information storage unit 121. The sensor information storage unit 121 may include a magnetic storage device such as a hard disk drive (HDD), a semiconductor storage device, an optical storage device, a magneto-optical storage device, or the like.

For example, as illustrated in FIG. 2, the peak position estimation unit 102 may estimate the peak position of the pulse waveform fitted by the fitting unit 101 as the peak position of the pulse wave signal. In this event, the peak position of the pulse wave signal is expressed by a positional relationship with the four sensors S₁ to S₄. For example, the peak position estimation unit 102 may estimate the peak position at time t₀ as a position where the sensor S₂ and the sensor S₃ are divided by 3:7, or may estimate the peak position at time t₁ as a position where the sensor S₃ and the sensor S₄ are divided by 1:1.

The pulse wave propagation speed derivation unit 103 derives a pulse wave propagation speed of the subject on the basis of the peak position estimated by the peak position estimation unit 102. Specifically, the pulse wave propagation speed derivation unit 103 can derive the pulse wave propagation speed of the subject by dividing a difference between the peak positions of the pulse wave signals at different times by a difference between times when the pulse wave signals are sensed.

The fitting of the predetermined pulse waveform to the pulse wave signals sensed by the plurality of sensors S₁ to S_{N} and the estimation of the peak position are performed at a first interval (for example, every 0.5 seconds to 1.0 seconds). Thus, the pulse wave propagation speed derivation unit 103 can derive the pulse wave propagation speed at a second interval (for example, every 10 seconds) longer than the first interval.

For example, as illustrated in FIG. 2, the pulse wave propagation speed derivation unit 103 can derive the pulse wave propagation speed by dividing a distance L between the estimated peak position at time t₀ and the estimated peak position at time t₁ by time t₀, time t₁, and a time difference (t₁−t₀).

A blood pressure derivation unit 400 derives a blood pressure of the subject on the basis of the derived pulse wave propagation speed. The blood pressure is affected by various factors such as a cardiac output pressure or stiffness of the artery, but can be calculated by a mathematical model using the pulse wave propagation speed particularly related to the stiffness of the artery and a constant for each subject. Note that the blood pressure derived from the pulse wave propagation speed is strictly a relative value. It is therefore desirable that the blood pressure derived from the pulse wave propagation speed is calibrated at a predetermined timing using a blood pressure value measured by a sphygmomanometer, or the like.

The information processing apparatus 100 according to the present embodiment having the above-described configuration can estimate the peak position of the pulse wave signal with higher accuracy by fitting a predetermined pulse waveform to the pulse wave signals sensed by the plurality of sensors S₁ to S_{N} on the same arterial path. With this arrangement, the information processing apparatus 100 can stabilize quality of the peak position estimation by fitting even in a case where signal quality of some sensors among the plurality of sensors S₁ to S_{N} is not high. Thus, the information processing apparatus 100 can derive the pulse wave propagation speed with high accuracy.

### 1.2. Operation Example of Information Processing Apparatus

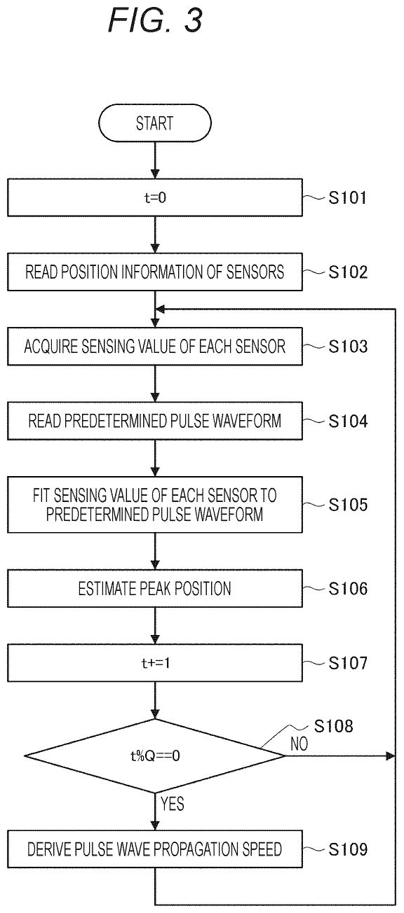

Next, an operation example of the information processing apparatus 100 according to the present embodiment will be described with reference to FIG. 3. FIG. 3 is a flowchart indicating flow of operation of the information processing apparatus 100.

In the flowchart indicated in FIG. 3, the peak position estimation of the pulse wave signal is performed every second, and the pulse wave propagation speed is derived every Q seconds (for example, Q=10). Note that, as Q is larger, the derived value of the pulse wave propagation speed is stabilized, but there is a possibility that fluctuation of the pulse wave propagation speed within a period of Q is overlooked. Thus, Q may be appropriately varied according to a purpose or a use situation.

As indicated in FIG. 3, first, after setting time t to 0 (S101), the information processing apparatus 100 reads information regarding the positions of the sensors S₁ to S_{N} from the sensor information storage unit 121 (S102). Next, the information processing apparatus 100 acquires a sensing value of each of the sensors S₁ to S_{N} (S103) and reads a predetermined pulse waveform from the pulse waveform storage unit 122 (S104).

Subsequently, the information processing apparatus 100 fits a predetermined pulse waveform to the sensing value of each of the sensors S₁ to S_{N} by the fitting unit 101 (S105). Next, in the information processing apparatus 100, the peak position estimation unit 102 estimates the peak position of the pulse wave signal from the fitted pulse waveform (S106).

Thereafter, the information processing apparatus 100 increments the count of the time t by 1 (S107) and then determines whether or not the remainder when the time t is divided by Q becomes 0 (S108). In a case where the remainder does not become 0 (S108: No), the information processing apparatus 100 returns to the operation of step S103 and estimates the peak position of the pulse wave signal at the next time.

On the other hand, in a case where the remainder becomes 0 (S108: Yes), the information processing apparatus 100 derives the pulse wave propagation speed using the peak position of the pulse wave signal estimated in the latest Q seconds (S109). By this means, the information processing apparatus 100 can derive the pulse wave propagation speed every Q seconds. Thereafter, the information processing apparatus 100 returns to the operation of step S103 and estimates the peak position of the pulse wave signal at the next time.

According to the above operation, the information processing apparatus 100 can estimate the peak position of the pulse wave signal every 1 second and derive the pulse wave propagation speed every Q seconds. The information processing apparatus 100 can stably estimate the peak position by fitting to a predetermined pulse waveform, so that it is possible to derive the pulse wave propagation speed with high accuracy.

### 1.3. Examples of Measurement Device

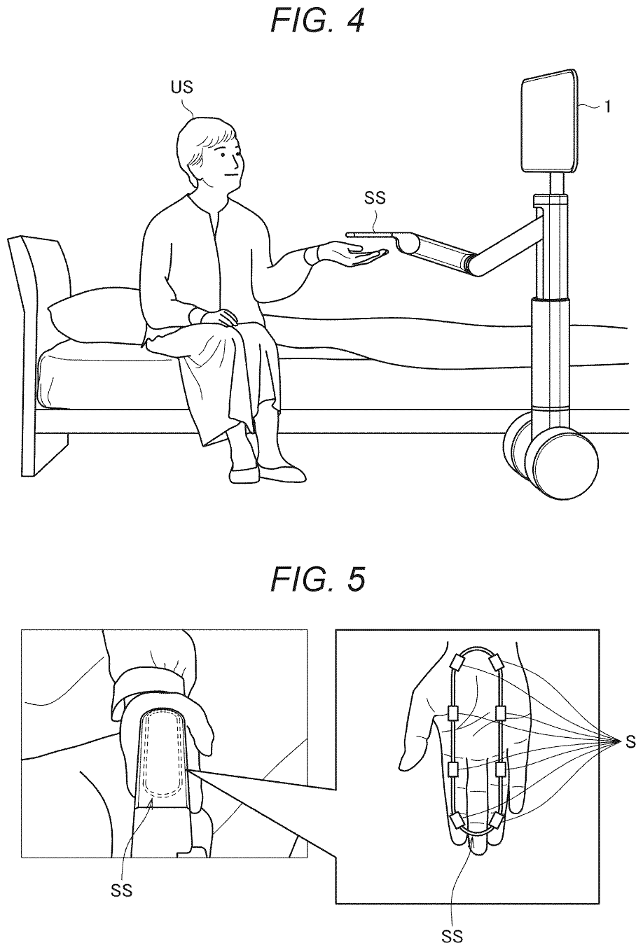

Next, first to sixth examples of the measurement device provided with the sensors S₁ to S_{N} (hereinafter, also collectively referred to as a sensor S) in the present embodiment will be described with reference to FIGS. 4 to 10.

### First Example

FIG. 4 is a schematic view illustrating an appearance of a measurement device 1 according to a first example. FIG. 5 is a schematic view illustrating an extracted measurement unit SS of the measurement device 1 according to the first example.

As illustrated in FIG. 4, the measurement device 1 according to the first example may be a robot device including a movement mechanism that autonomously moves the measurement device 1, a display device that displays an instruction, or the like, to a subject US for whom a blood pressures is to be measured, and the measurement unit SS including the sensor S.

As illustrated in FIG. 5, the sensor S that senses the pulse wave of the subject US is disposed on a back surface of the measurement unit SS in a direction from the wrist of the subject US toward the hand. The measurement device 1 can sense the pulse wave of the subject US by the sensor S on the back surface of the measurement unit SS by transmitting an instruction to the subject US so as to hold the hand over the back surface of the measurement unit SS through the image displayed on the display device.

This enables the measurement device 1 to sense the pulse wave at at least three or more different positions on the same arterial path from the wrist to the hand, so that the pulse wave propagation speed and the blood pressure of the subject US can be measured. The measurement device 1 can move autonomously by the movement mechanism, so that the measurement device 1 can move near the subject US and measure the blood pressure of the subject US when it is necessary to measure the blood pressure.

### Second Example

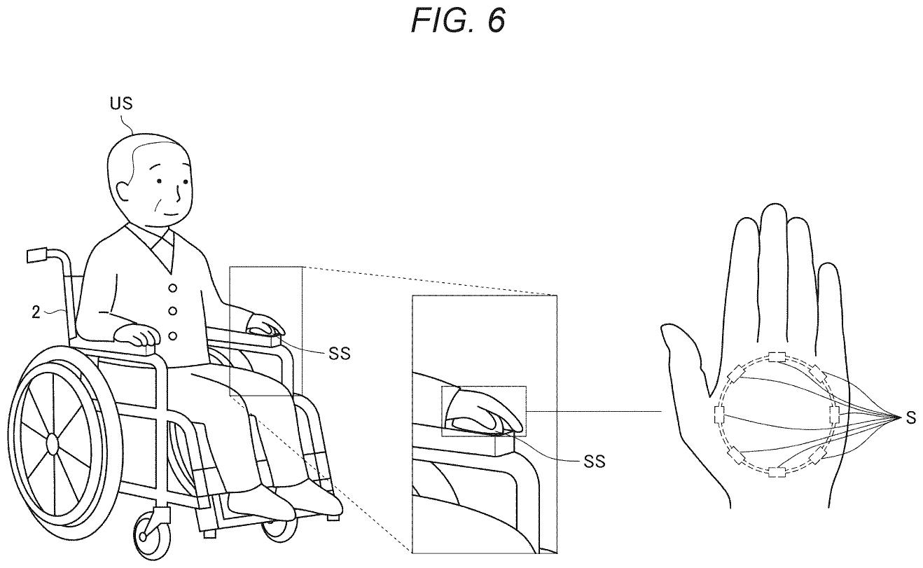

FIG. 6 is a schematic view illustrating an appearance of a measurement device 2 according to a second example.

As illustrated in FIG. 6, the measurement device 2 according to the second example may be an electric wheelchair to be used by the subject US for whom the blood pressure is to be measured. Specifically, the measurement unit SS including the sensor S may be provided, for example, in a controller unit for the subject US to input a movement instruction to the electric wheelchair. In the measurement unit SS, the sensor S that senses the pulse wave of the subject US is disposed at positions overlapping the palm of the subject US in a ring shape.

This enables the measurement device 2 to sense the pulse wave at at least three or more different positions on the same arterial path from the wrist to the hand, so that the pulse wave propagation speed and the blood pressure of the subject US can be measured. The measurement device 2 can measure the blood pressure of the subject US who is using the electric wheelchair at an arbitrary timing by providing the measurement unit SS at the portion where the subject US places the palm when using the electric wheelchair.

### Third Example

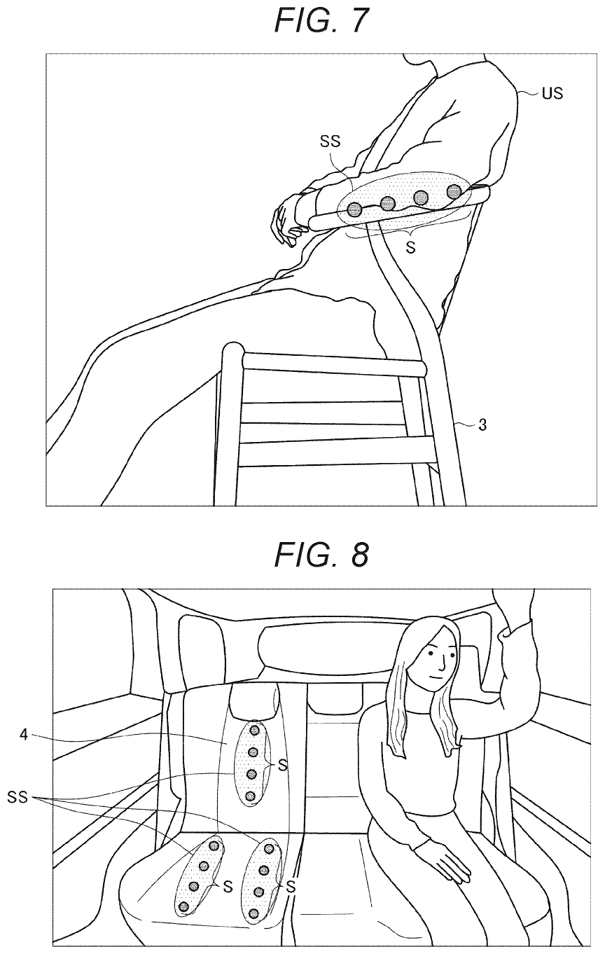

FIG. 7 is a schematic view illustrating an appearance of a measurement device 3 according to a third example.

As illustrated in FIG. 7, the measurement device 3 according to the third example may be a chair to be used by the subject US for whom the blood pressure is to be measured. Specifically, the measurement unit SS including the sensor S may be provided on an armrest portion of the chair. In the measurement unit SS, the sensor S that senses the pulse wave of the subject US is disposed along an extending direction of the armrest portion. Note that, in the measurement device 3 according to the third example, the pulse wave of the subject US is sensed from above the clothes. Thus, the sensor S may be a sensor that physically detects pulsation, such as an acceleration sensor or a pressure sensor, or a microwave Doppler sensor that can detect heart rate variability in a state where the subject US wears the clothes.

This enables the measurement device 3 to sense the pulse wave at at least three or more different positions on the same arterial path from the shoulder to the hand, so that the pulse wave propagation speed and the blood pressure of the subject US can be measured. The measurement device 3 can measure the blood pressure of the subject US who is using the chair at an arbitrary timing by providing the measurement unit SS at the portion where the elbow is placed when the subject US uses the chair.

### Fourth Example

FIG. 8 is a schematic view illustrating an appearance of a measurement device 4 according to a fourth example.

As illustrated in FIG. 8, the measurement device 4 according to the fourth example may be a seat of an automobile, or the like, to be used by the subject US for whom the blood pressure is to be measured. Specifically, the measurement unit SS including the sensor S may be provided in a backrest portion or a seating portion. In the measurement unit SS, the sensor S that senses the pulse wave of the subject US is disposed along an extending direction of the backrest portion or the seating portion. Note that, in the measurement device 4 according to the fourth example, the pulse wave of the subject US is sensed from above the clothes, similarly to the third example. Thus, the sensor S may be a sensor that physically detects pulsation, such as an acceleration sensor or a pressure sensor, or a microwave Doppler sensor that can detect heart rate variability in a state where the subject US wears the clothes.

This enables the measurement device 4 to sense the pulse wave at at least three or more different positions of the same arterial path from the heart to the terminal, so that the pulse wave propagation speed and the blood pressure of the subject US can be measured. In addition, the measurement device 4 is provided as a seat to be used when the subject US drives the automobile, so that it is possible to monitor the blood pressure of the subject US who is driving the automobile at an arbitrary timing.

### Fifth Example

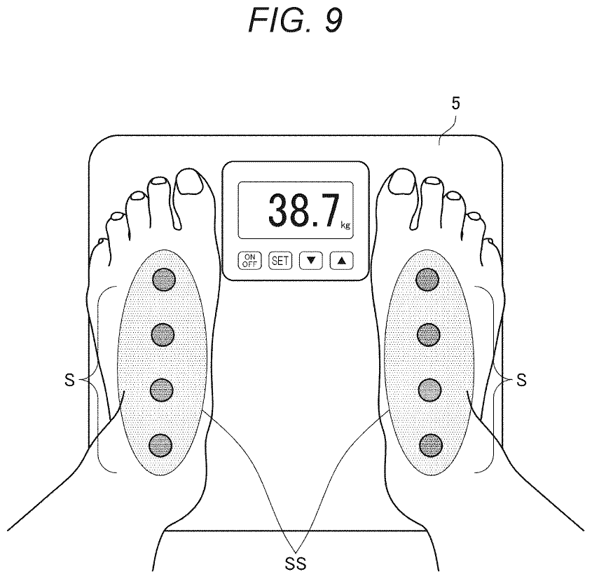

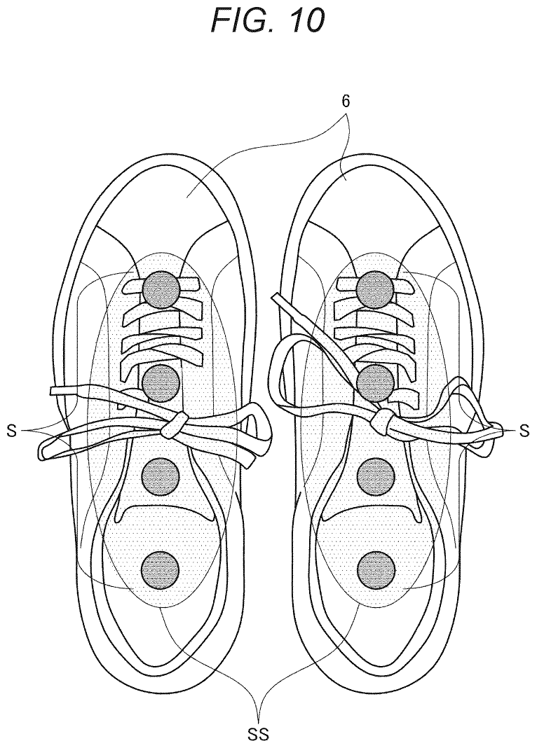

FIG. 9 is a schematic view illustrating an appearance of a measurement device 5 according to a fifth example.

As illustrated in FIG. 9, the measurement device 5 according to the fifth example may be a scale to be used by the subject US for whom the blood pressure is to be measured. Specifically, the measurement unit SS including the sensor S may be provided on a contact foot surface with which the foot of the subject US comes into contact. In the measurement unit SS, the sensor S that senses the pulse wave of the subject US in a predetermined direction is disposed.

This enables the measurement device 5 to sense the pulse wave at at least three or more different positions on the same arterial path from the heel to the toe, so that the pulse wave propagation speed and the blood pressure of the subject US can be measured. The measurement device 5 can simultaneously measure the blood pressure of the subject US when the subject US measures the body weight.

### Sixth Example

FIG. 10 is a schematic view illustrating an appearance of a measurement device 6 according to a sixth example.

As illustrated in FIG. 10, the measurement device 6 according to the sixth example may be a shoe to be used by the subject US for whom the blood pressure is to be measured or an insole inserted into the shoe. Specifically, the measurement unit SS including the sensor S may be provided on a bottom surface with which the sole of the foot of the subject US is in contact, or may be provided on a surface with which the top of the foot of the subject US is in contact. In the measurement unit SS, the sensor S that senses the pulse wave of the subject US from the heel toward the toe of the shoe is disposed.

This enables the measurement device 6 to sense the pulse wave at at least three or more different positions on the same arterial path from the heel to the toe, so that the pulse wave propagation speed and the blood pressure of the subject US can be measured. The measurement device 6 can monitor the blood pressure of the subject US at an arbitrary timing when the subject US goes out.

### 2. SECOND EMBODIMENT

### 2.1. Configuration Example of Information Processing Apparatus

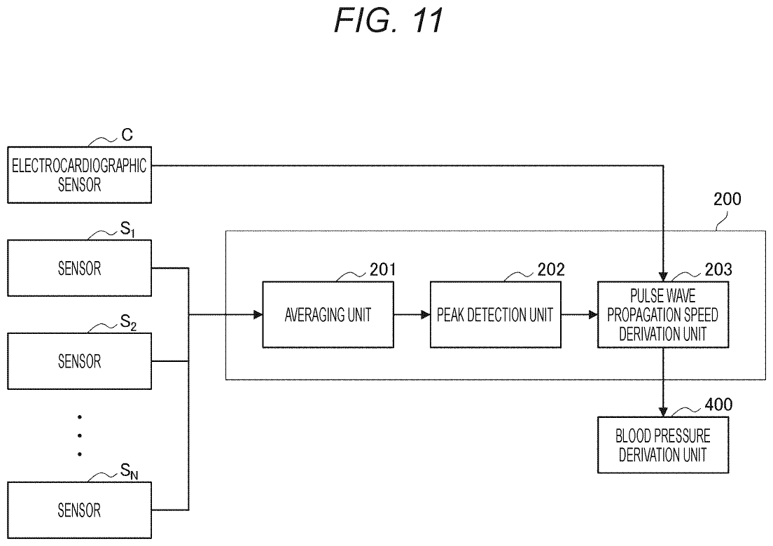

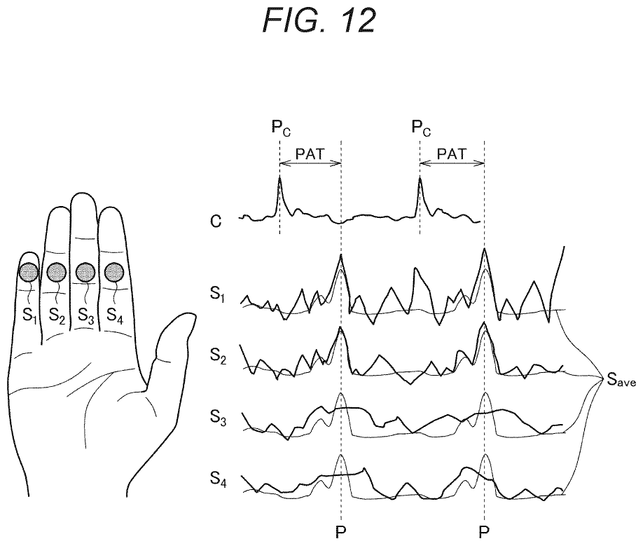

Next, a configuration of an information processing apparatus according to a second embodiment of the present disclosure will be described with reference to FIGS. 11 and 12. FIG. 11 is a block diagram illustrating a functional configuration of the information processing apparatus 200 according to the present embodiment. FIG. 12 is an explanatory diagram for explaining a specific example of processing by the information processing apparatus 200.

As illustrated in FIG. 11, the information processing apparatus 200 includes an averaging unit 201, a peak detection unit 202, and a pulse wave propagation speed derivation unit 203. The information processing apparatus 200 can derive an average pulse wave signal by averaging each of the pulse wave signals sensed by the plurality of sensors S₁ to S_{N} and derive a pulse wave propagation speed on the basis of a time difference between a peak of the average pulse wave signal and an electrocardiogram peak.

The averaging unit 201 derives an average pulse wave signal by averaging each of the pulse wave signals sensed by the plurality of sensors S₁ to S_{N}. Specifically, the averaging unit 201 derives an average pulse wave signal by averaging each of values of the pulse wave signals sensed in the same time section by the plurality of sensors S₁ to S_{N}.

Similarly to the first embodiment, the plurality of sensors S₁ to S_{N} is a sensor that optically senses pulse wave on the body surface of the subject. The plurality of sensors S₁ to S_{N} can sense the pulse wave at at least three or more positions at substantially the same distance from the heart of the subject, thereby reducing influence of the signal quality of each of the plurality of sensors S₁ to S_{N} on the average pulse wave signal. In order to further improve accuracy of the average pulse wave signal, the number of sensors S₁ to S_{N} is desirably three or more. An upper limit of the number of the sensors S₁ to S_{N} is not particularly limited, but may be, for example, 10 from the viewpoint of cost and installation location. Note that substantially the same distance from the heart means that the peak of the pulse wave reaches at the same time. For example, the body surface having substantially the same distance from the heart may be each finger of the hand.

For example, as illustrated in FIG. 12, in a case where four sensors S₁ to S₄ are provided for each of the little finger, the ring finger, the middle finger, and the index finger of the subject, it is considered that pulse wave signals having substantially the same waveform are sensed in the four sensors S₁ to S₄ because the distances from the heart are substantially the same. In such a case, the averaging unit 201 can cancel noise included in each of the pulse wave signals by averaging the pulse wave signals sensed by the four sensors S₁ to S₄. Thus, the averaging unit 201 can derive the average pulse wave signal Sₐᵥₑ from which the peak is more easily detected by reducing the influence of noise.

The peak detection unit 202 detects the peak of the average pulse wave signal derived by the averaging unit 201. Specifically, the peak detection unit 202 may detect a convex portion having signal intensity equal to or higher than a predetermined value in the average pulse wave signal as a pulsation peak and detect the time of the peak.

For example, as illustrated in FIG. 12, the peak detection unit 202 may detect a peak time p of the average pulse wave signal Sₐᵥₑ derived by the averaging unit 201. In the average pulse wave signal, noise included in each of the pulse wave signals detected by the four sensors S₁ to S₄ is canceled by averaging. Thus, the peak detection unit 202 can more easily detect the peak from the average pulse wave signal with reduced noise.

The pulse wave propagation speed derivation unit 203 derives the pulse wave propagation speed of the subject on the basis of the peak detected by the peak detection unit 202 and the electrocardiogram peak. Specifically, the pulse wave propagation speed derivation unit 203 derives the pulse wave propagation speed of the subject on the basis of a difference between the time of the electrocardiogram peak detected by the electrocardiographic sensor C and the peak time detected by the peak detection unit 202. For example, the pulse wave propagation speed derivation unit 203 can derive the pulse wave propagation speed of the subject by dividing distances from the heart to the sensing positions by the plurality of sensors S₁ to S_{N} by the difference between the time of the electrocardiogram peak and the detected peak time.

Note that, in a case where the sensing positions by the plurality of sensors S₁ to S_{N} are fixed, the distances from the heart to the sensing positions by the plurality of sensors S1 to S_{N} become a constant. In such a case, the pulse wave propagation speed derivation unit 203 may derive the pulse wave propagation speed as a relative value without using the distances from the heart to the sensing positions by the plurality of sensors S₁ to S_{N}.

The electrocardiogram peak is a peak of pulsation detected from the electrocardiogram measured by the electrocardiogram sensor C. The electrocardiographic sensor C is a sensor that measures a state of electrical activity of the heart by electrodes attached to the body surface at positions sandwiching the heart of the subject. In other word, the pulse wave propagation speed derivation unit 203 can derive the pulse wave propagation speed of the subject on the basis of periods until the electrocardiogram peak measured by the electrocardiographic sensor C reaches the sensing positions by the plurality of sensors S₁ to S_{N}.

For example, as illustrated in FIG. 12, the pulse wave propagation speed derivation unit 203 may derive the pulse wave propagation speed from a time difference PAT between the electrocardiogram peak P_{c} and the peak time P of the average pulse wave signal Sₐᵥₑ.

A blood pressure derivation unit 400 derives a blood pressure of the subject on the basis of the derived pulse wave propagation speed. The blood pressure is affected by various factors such as a cardiac output pressure or stiffness of the artery, but can be calculated by a mathematical model using the pulse wave propagation speed particularly related to the stiffness of the artery and a constant for each subject. Note that the blood pressure derived from the pulse wave propagation speed is strictly a relative value. It is therefore desirable that the blood pressure derived from the pulse wave propagation speed is calibrated at a predetermined timing using a blood pressure value measured by a sphygmomanometer, or the like.

The information processing apparatus 200 according to the present embodiment having the above configuration can detect the peak of the pulse wave signal with higher accuracy by averaging the pulse wave signals sensed by the plurality of sensors S₁ to S_{N} having substantially the same distance from the heart. By this means, even in a case where the signal quality of the plurality of sensors S₁ to S_{N} is not high, the information processing apparatus 200 can stably detect the peak by averaging.

Thus, the information processing apparatus 200 can derive the pulse wave propagation speed with high accuracy.

### 2.2. Operation Example of Information Processing Apparatus

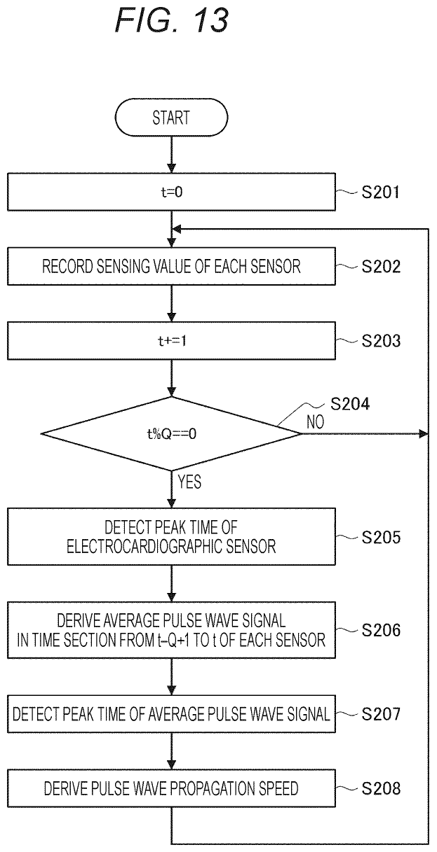

Next, an operation example of the information processing apparatus 200 according to the present embodiment will be described with reference to FIG. 13. FIG. 13 is a flowchart indicating flow of operation of the information processing apparatus 200.

In the flowchart indicated in FIG. 13, the pulse wave propagation speed is derived every Q seconds (for example, Q=10). Note that, as Q is larger, the derived value of the pulse wave propagation speed is stabilized, but there is a possibility that fluctuation of the pulse wave propagation speed within a period of Q is overlooked. Thus, Q may be appropriately varied according to a purpose or a use situation.

As indicated in FIG. 13, first, after setting the time t to 0 (S201), the information processing apparatus 200 records a sensing value of each of the sensors S₁ to S_{N} in a memory, or the like (S202).

Thereafter, the information processing apparatus 200 increments the count of the time t by 1 (S203) and then determines whether or not the remainder when the time t is divided by Q becomes 0 (S204). In a case where the remainder does not become 0 (S204: No), the information processing apparatus 200 returns to the operation of step S202 and records the sensing value of each of the sensors S₁ to S_{N} at the next time in the memory, or the like.

On the other hand, in a case where the remainder becomes 0 (S204: Yes), the information processing apparatus 200 detects the time of the electrocardiogram peak of the electrocardiogram sensor C (S205). Next, the information processing apparatus 200 derives an average pulse wave signal by averaging, by the averaging unit 201, pulse wave signals sensed by each of the sensors S₁ to S_{N} in a time section (that is, a time section of the last Q seconds) from time t−Q+1 to the time t (S206).

Subsequently, the information processing apparatus 200 detects the peak time of the average pulse wave signal by detecting the peak of the average pulse wave signal by the peak detection unit 202 (S207). Thereafter, in the information processing apparatus 200, the pulse wave propagation speed derivation unit 203 derives the pulse wave propagation speed using a difference between the time of the electrocardiogram peak and the peak time of the average pulse wave signal (S208).

This enables the information processing apparatus 200 to derive the pulse wave propagation speed every Q seconds. Thereafter, the information processing apparatus 200 returns to the operation of step S202 and records a sensing value of each of the sensors S₁ to S_{N} at the next time in the memory, or the like.

According to the above operation, the information processing apparatus 200 can derive the pulse wave propagation speed every Q seconds by averaging the pulse wave signals for the latest Q seconds. The information processing apparatus 200 can stably detect the peak by averaging the pulse wave signals sensed by each of the sensors S₁ to S_{N}, and thus can derive the pulse wave propagation speed with high accuracy.

### 2.3. Example of Measurement Device

Next, with reference to FIGS. 14 to 18, first to fifth examples of the measurement device provided with the sensors S₁ to S_{N} (hereinafter, also collectively referred to as the sensor S) in the present embodiment will be described.

### First Example

FIG. 14 is a schematic view illustrating an appearance of a measurement device 7 according to a first example.

As illustrated in FIG. 14, the measurement device 7 according to the first example may be a steering wheel of an automobile to be driven by the subject US for whom the blood pressure is to be measured. Specifically, the measurement unit SS including the sensor S and the electrocardiogram sensor C may be provided at a position where the steering wheel is to be held by the subject US. For example, the sensor S that senses the pulse wave may be disposed at positions corresponding to the respective fingers of the subject US in the measurement unit SS on a back side as viewed from the subject US who is the driver. In addition, the electrocardiographic sensor C that acquires an electrocardiogram may be disposed at a position corresponding to the palm of the subject US in the measurement unit SS on a front side as viewed from the subject US who is the driver.

This enables the measurement device 7 to acquire the electrocardiogram of the subject US and sense the pulse wave of the subject US at the respective fingers, so that it is possible to measure the pulse wave propagation speed and the blood pressure of the subject US. In addition, the measurement device 7 is provided as a steering wheel to be held by the subject US when the subject US drives the automobile, so that it is possible to monitor the blood pressure of the subject US who is driving the automobile at an arbitrary timing.

### Second Example

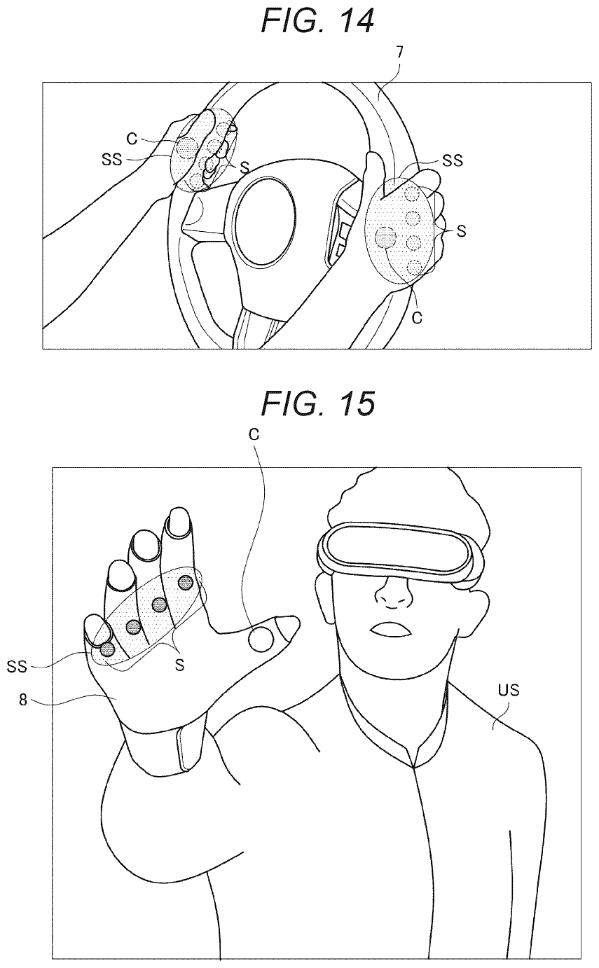

FIG. 15 is a schematic view illustrating an appearance of a measurement device 8 according to a second example.

As illustrated in FIG. 15, the measurement device 8 according to the second example may be a glove to be worn on both hands of the subject US for whom the blood pressure is to be measured. The glove that functions as the measurement device 8 may be a glove that operates as an augmented reality (AR) controller, or may be a glove worn during sports or work. Specifically, the measurement unit SS including the sensor S and the electrocardiographic sensor C may be provided inside the globe so as to correspond the respective fingers. For example, the sensor S that senses pulse wave may be disposed inside the glove so as to correspond to each of the index finger, the middle finger, the ring finger, and the little finger of the subject US. In addition, the electrocardiographic sensor C that acquires an electrocardiogram may be disposed inside the glove so as to correspond to the thumb of the subject US.

This enables the measurement device 8 to acquire the electrocardiogram of the subject US and sense the pulse wave of the subject US at the respective fingers, so that it is possible to measure the pulse wave propagation speed and the blood pressure of the subject US. In addition, the measurement device 8 is provided as a glove to be worn by the subject US at the time of activities such as AR activity, sports, or work, so that the blood pressure of the subject US during these activities can be monitored at an arbitrary timing.

### Third Example

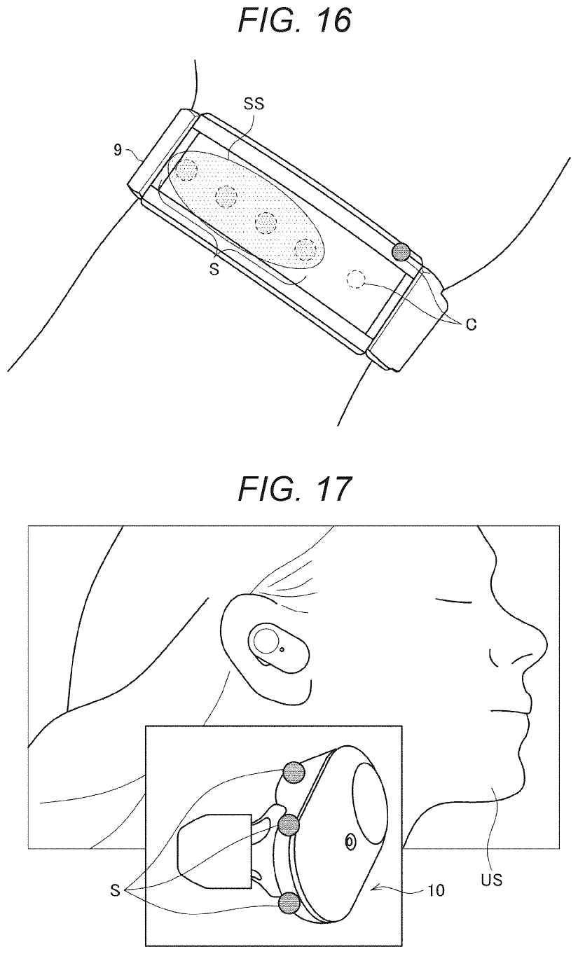

FIG. 16 is a schematic view illustrating an appearance of a measurement device 9 according to a third example.

As illustrated in FIG. 16, the measurement device 9 according to the third example may be a smart watch to be worn by the subject US for whom the blood pressure is to be measured. Specifically, the measurement unit SS including the sensor S and the electrocardiogram sensor C may be provided on a back side of the smartwatch that comes into contact with the wrist of the subject US. For example, the sensor S may be provided on the back side of the smartwatch along a circumferential direction of the wrist of the subject US. In addition, the electrocardiogram sensor C may be provided on each of the back side and the side surface of the smart watch. The subject US can cause the measurement device 9 to perform electrocardiogram measurement by touching one electrode of the electrocardiogram sensor C provided on the side surface with the hand opposite to the hand on which the measurement device 9 is worn (that is, the hand with which the other electrode of the electrocardiographic sensor C provided on the back surface is in contact).

This enables the measurement device 9 to acquire the electrocardiogram of the subject US and sense the pulse wave of the subject US at the respective fingers, so that it is possible to measure the pulse wave propagation speed and the blood pressure of the subject US. In addition, the measurement device 9 is provided as a smart watch to be worn by the subject US on a daily basis, so that the blood pressure of the subject US can be monitored on a daily basis.

### Fourth Example

FIG. 17 is a schematic view illustrating an appearance of a measurement device 10 according to a fourth example.

As illustrated in FIG. 17, the measurement device 10 according to the fourth example may be an earphone to be worn by the subject US for whom the blood pressure is to be measured. Specifically, the sensor S of the measurement device 10 may be provided at a portion that comes into contact with the auricle of the earphone. Note that in the fourth example, an electrocardiogram is separately acquired by an electrocardiographic sensor (not illustrated).

This enables the measurement device 10 to measure the pulse wave propagation speed and the blood pressure of the subject US on the basis of the electrocardiogram separately acquired and the pulse wave of the subject US sensed by each auricle. In addition, the measurement device 10 can simultaneously measure the blood pressure of the subject US together with sound output by the earphone.

### Fifth Example

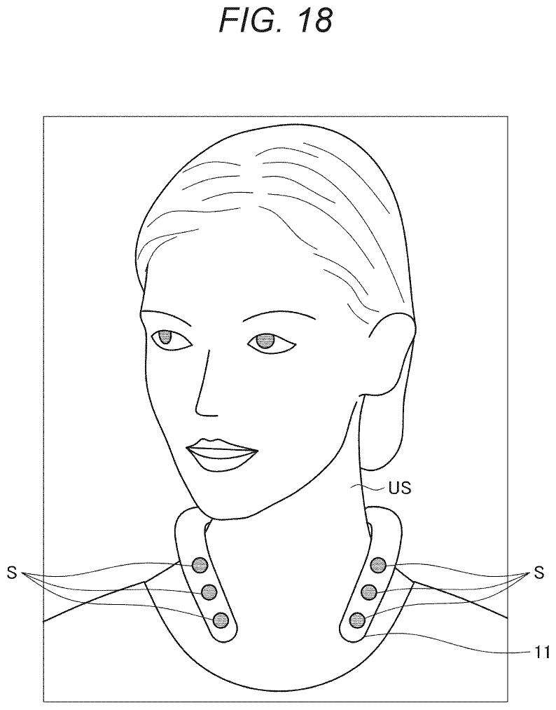

FIG. 18 is a schematic view illustrating an appearance of a measurement device 11 according to a fifth example.

As illustrated in FIG. 18, the measurement device 11 according to the fifth example may be a neck band speaker to be worn by the subject US for whom the blood pressure is to be measured. Specifically, the sensor S of the measurement device 11 may be provided on a back side of the neck band that comes into contact with the body surface of the subject US. Note that in the fifth example, an electrocardiogram is separately acquired by an electrocardiographic sensor (not illustrated).

This enables the measurement device 11 to measure the pulse wave propagation speed and the blood pressure of the subject US on the basis of the separately acquired electrocardiogram and the pulse wave of the subject US sensed around the neck. In addition, the measurement device 11 can simultaneously measure the blood pressure of the subject US together with sound output by the neck band speaker.

### 3. THIRD EMBODIMENT

### 3.1. Configuration Example of Information Processing Apparatus

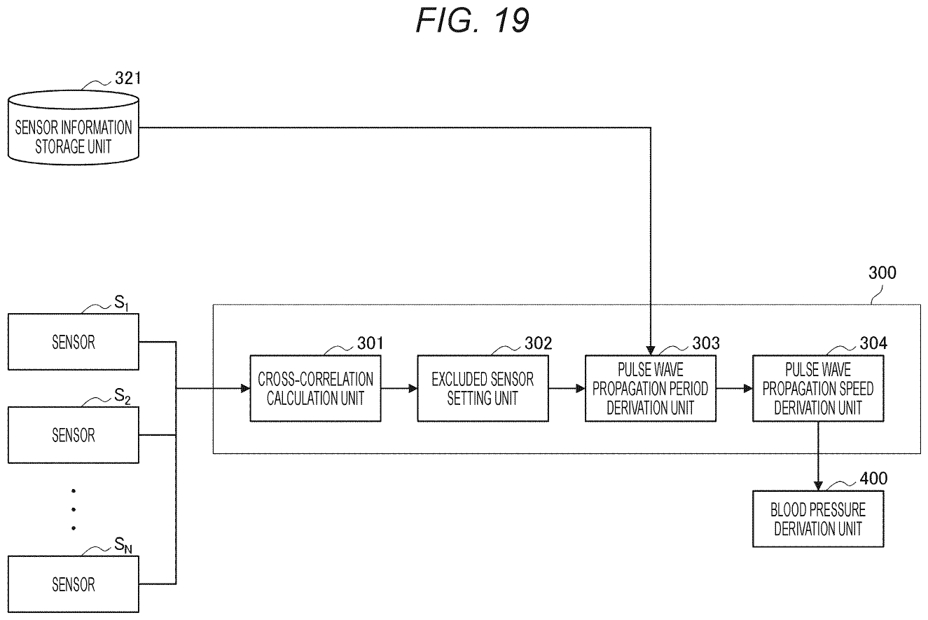

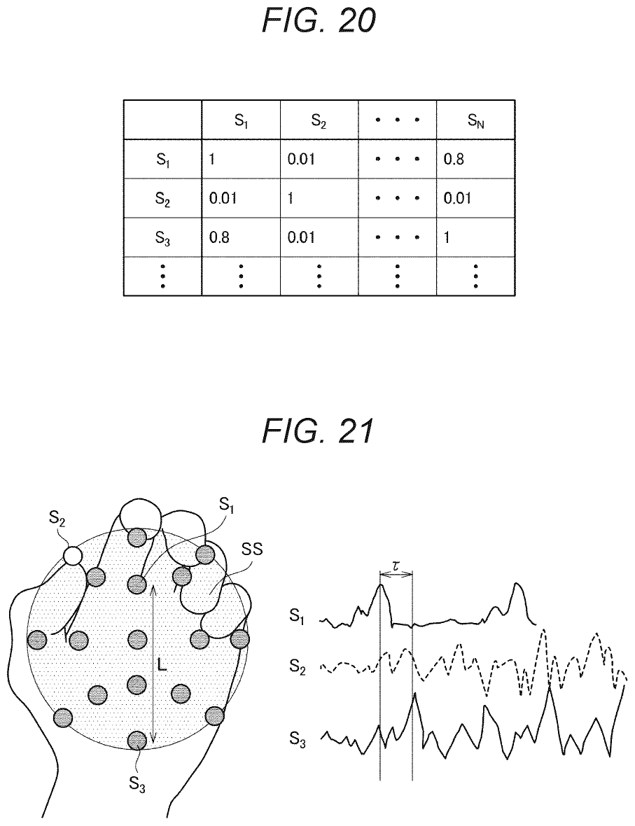

Next, a configuration of an information processing apparatus according to a third embodiment of the present disclosure will be described with reference to FIGS. 19 to 21. FIG. 19 is a block diagram illustrating a functional configuration of an information processing apparatus 300 according to the present embodiment. FIGS. 20 and 21 are explanatory diagrams for explaining a specific example of processing by the information processing apparatus 300.

As illustrated in FIG. 19, the information processing apparatus 300 includes a cross-correlation calculation unit 301, an excluded sensor setting unit 302, a pulse wave propagation period derivation unit 303, and a pulse wave propagation speed derivation unit 304. The information processing apparatus 300 calculates each of cross-correlations of the pulse wave signals sensed by the plurality of sensors S₁ to S_{N}, thereby being able to derive the pulse wave propagation speed using only the pulse wave signals having the calculated cross-correlations equal to or higher than a threshold.

The cross-correlation calculation unit 301 calculates each of cross-correlations of the pulse wave signals sensed by the plurality of sensors S₁ to S_{N}. Specifically, the cross-correlation calculation unit 301 calculates each of cross-correlation functions of the pulse wave signals sensed in the same time section by the plurality of sensors S₁ to S_{N} and further calculates maximum values of absolute values of the calculated cross-correlation functions. This enables the cross-correlation calculation unit 301 to generate data in which maximum values of absolute values of cross-correlation functions between each of the plurality of sensors S₁ to S_{N} and other sensors are arranged in a matrix as indicated in FIG. 20.

Similarly to the first embodiment, the plurality of sensors S₁ to S_{N} is a sensor that optically senses pulse wave on the body surface of the subject. As illustrated in FIG. 21, the plurality of sensors S₁ to S_{N} is provided at arbitrary positions of the measurement unit SS.

Here, in a case where the measurement unit SS comes into contact with the body surface of the subject at an arbitrary position and posture, it may be difficult to favorably sense the pulse wave from the body surface of the subject with all the plurality of sensors S₁ to S_{N}. This is because, depending on a positional relationship between the plurality of sensors S₁ to S_{N} and the body surface of the subject, it is difficult for all of the plurality of sensors S₁ to S_{N} to be put into a state in contact with the body surface of the subject, in which favorable sensing is possible.

The information processing apparatus 300 can exclude a sensor that has a low cross-correlation with other sensors and is determined to have not favorably sensed the pulse wave, and derive the pulse wave propagation speed using only the sensors that are determined to have favorably sensed the pulse wave. This enables the information processing apparatus 300 to exclude a pulse wave signal having low signal quality, so that it is possible to derive the pulse wave propagation speed with higher accuracy. Note that in order to derive the pulse wave propagation speed with higher accuracy, the number of sensors S₁ to S_{N} is desirably three or more. An upper limit of the number of the sensors S₁ to S_{N} is not particularly limited, but may be, for example, 20 from the viewpoint of cost and installation location.

The excluded sensor setting unit 302 sets a sensor that is not to be used for deriving the pulse wave propagation speed on the basis of the cross-correlations of the plurality of sensors S₁ to S_{N}. Specifically, the excluded sensor setting unit 302 sets a sensor in which the maximum values of the absolute values of the cross-correlation functions with other sensors calculated by the cross-correlation calculation unit 301 are all less than the threshold as a sensor that is not to be used for deriving the pulse wave propagation speed. This is because, in a sensor that favorably senses the pulse wave of the subject, a cross-correlation due to the peak of the pulse wave occurs, and thus, it is considered that a sensor having low cross-correlation with other sensors has not favorably sensed the pulse wave.

In the example indicated in FIG. 20, the excluded sensor setting unit 302 may set the sensor S₂ in which the maximum values of the absolute values of the cross-correlation functions with other sensors are all less than the threshold as a sensor that is not to be used for deriving the sensor pulse wave propagation speed. In such a case, as illustrated in FIG. 21, it is considered that the sensor S₂ has no contact with the body surface of the subject and has not been able to favorably sense the peak of the pulse wave. On the other hand, the excluded sensor setting unit 302 may set the sensors S₁ and S₃ in which any of the maximum values of the absolute values of the cross-correlation functions with the other sensors is equal to or more than the threshold as the sensors to be used for deriving the pulse wave propagation speed.

The pulse wave propagation period derivation unit 303 derives a pulse wave propagation period between the sensors excluding the sensor set not to be used for deriving the pulse wave propagation speed. Specifically, the pulse wave propagation period derivation unit 303 derives a difference in timing at which the maximum values of the cross-correlation functions are obtained among the sensors excluding the sensor set not to be used for deriving the pulse wave propagation speed. As a result, the pulse wave propagation period derivation unit 303 can derive the difference in timing at which the cross-correlation functions become the maximum values as the pulse wave propagation period between the sensors. This is because, in the pulse wave signals, a cross-correlation due to the peak of the pulse wave occurs, and thus, the timing at which the cross-correlation function becomes the maximum value is considered to be the timing at which the pulse wave becomes the peak.

For example, as illustrated in FIG. 21, the pulse wave propagation period derivation unit 303 may detect a difference (that is, deviation of peak timing) τ between timings at which the maximum values of the cross-correlation functions are obtained for the sensor S₁ and the sensor S₃ excluding the sensor S₂ set not to be used for deriving the pulse wave propagation speed. The pulse wave propagation period derivation unit 303 can derive the detected deviation τ as the pulse wave propagation period between the sensor S₁ and the sensor S₃.

The pulse wave propagation speed derivation unit 304 derives the pulse wave propagation speed of the subject on the basis of the pulse wave propagation period derived by the pulse wave propagation period derivation unit 303 and the distance between the sensors. Specifically, the pulse wave propagation speed derivation unit 304 derives the pulse wave propagation speed in each combination of the sensors by dividing the pulse wave propagation period by the distance between the sensors for each combination of the sensors excluding the sensor set not to be used for deriving the pulse wave propagation speed. Furthermore, the pulse wave propagation speed derivation unit 304 can derive the pulse wave propagation speed as a whole by averaging each of the derived pulse wave propagation speeds.

The distance between the plurality of sensors S₁ to S_{N} may be expressed by the distance between each other on the arterial path. For example, in a case where the plurality of sensors S₁ to S_{N} is provided on a substantially linear arterial path, the distance between the plurality of sensors S₁ to S_{N} may be expressed by a linear distance between each other in a direction along the arterial path. In addition, in a case where the plurality of sensors S₁ to S_{N} is provided on the branched arterial path, the distance between the plurality of sensors S₁ to S_{N} may be expressed by a distance difference from a branch point of the arterial path. Information regarding the distance between the plurality of sensors S₁ to S_{N} is stored in advance in the sensor information storage unit 321. The sensor information storage unit 321 may include a magnetic storage device such as a hard disk drive (HDD), a semiconductor storage device, an optical storage device, a magneto-optical storage device, or the like.

For example, as illustrated in FIG. 21, the pulse wave propagation speed derivation unit 304 can derive the pulse wave propagation speed between the sensor S₁ and the sensor S₃ by dividing the distance L between the sensor S₁ and the sensor Sw by the difference τ (that is, pulse wave propagation period) between the peak timings of the sensor S₁ and the sensor S₃. The pulse wave propagation speed derivation unit 304 can derive the pulse wave propagation speed as a whole by deriving the pulse wave propagation speeds as described above also for sensors other than the sensor S₂ set not to be used for deriving the pulse wave propagation speed and by averaging the derived pulse wave propagation speeds.

Note that the pulse wave propagation speed derivation unit 304 may perform averaging by weighting when averaging the pulse wave propagation speeds derived for respective combinations of the sensors.

Specifically, the pulse wave propagation speed derivation unit 304 may perform averaging by giving a larger weight to the pulse wave propagation speed derived from a combination of sensors having a longer distance between the sensors.

Furthermore, the pulse wave propagation speed derivation unit 304 can also consider influence of gravity when deriving the pulse wave propagation speed for each combination of sensors. For example, in a direction in which gravity acts, the pulse wave propagation speed increases due to the influence of gravity. On the other hand, in a direction opposite to the direction in which gravity acts, the pulse wave propagation speed decreases due to the influence of gravity. Thus, in order to eliminate the influence of gravity, the pulse wave propagation speed derivation unit 304 may select a combination of sensors existing on the same horizontal plane and derive the pulse wave propagation speed. Alternatively, for a combination of sensors existing in the direction in which gravity acts, the pulse wave propagation speed derivation unit 304 may derive the pulse wave propagation speed by correcting the influence of gravity. The direction of gravity acting on each of the sensors can be detected by, for example, an acceleration sensor or a gyro sensor provided in the measurement unit SS.

The blood pressure derivation unit 400 derives a blood pressure of the subject on the basis of the derived pulse wave propagation speed. The blood pressure is affected by various factors such as a cardiac output pressure or stiffness of the artery, but can be calculated by a mathematical model using the pulse wave propagation speed particularly related to the stiffness of the artery and a constant for each subject. Note that the blood pressure derived from the pulse wave propagation speed is strictly a relative value. Thus, the blood pressure derived from the pulse wave propagation speed is desirably calibrated at a predetermined timing with the blood pressure value measured using a sphygmomanometer, or the like.

The information processing apparatus 300 according to the present embodiment having the above-described configuration can derive the pulse wave propagation speed by selectively using sensors whose cross-correlations are equal to or greater than the threshold and which are assumed to favorably sense a pulse wave. By this means, even in a case where the signal quality of the pulse wave signals sensed by the plurality of sensors S₁ to S_{N} is unstable, the information processing apparatus 300 can extract sensors that favorably sense the pulse wave signals and derive the pulse wave propagation speed. Thus, the information processing apparatus 300 can derive the pulse wave propagation speed with high accuracy.

### 3.2. Operation Example of Information Processing Apparatus

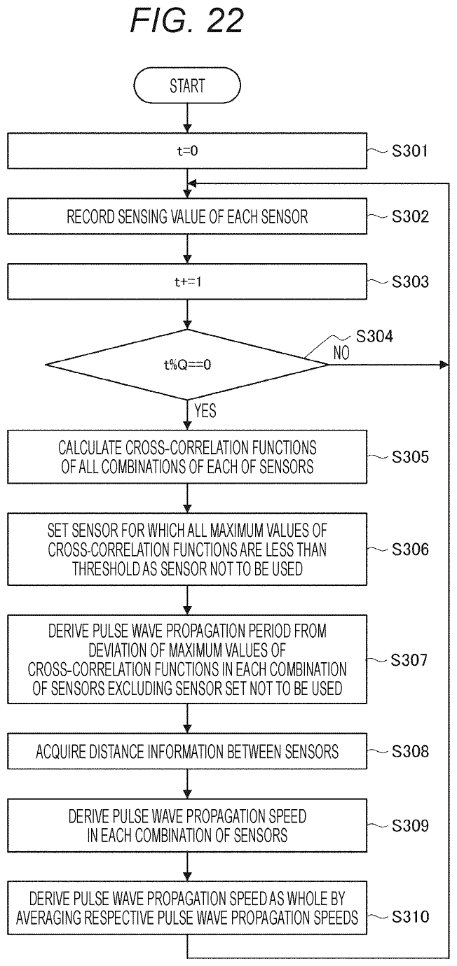

Next, an operation example of the information processing apparatus 300 according to the present embodiment will be described with reference to FIG. 22. FIG. 22 is a flowchart indicating flow of operation of the information processing apparatus 300.

In the flowchart indicated in FIG. 22, the pulse wave propagation speed is derived every Q seconds (for example, Q=10). Note that, as Q is larger, the derived value of the pulse wave propagation speed is stabilized, but there is a possibility that fluctuation of the pulse wave propagation speed within a period of Q is overlooked. Thus, Q may be appropriately varied according to a purpose or a use situation.

As indicated in FIG. 22, first, after setting the time t to 0 (S301), the information processing apparatus 300 records the sensing values of each of the sensors S₁ to S_{N} in the memory, or the like (S302).

Thereafter, the information processing apparatus 300 increments the count of the time t by 1 (S303) and then determines whether or not the remainder when the time t is divided by Q becomes 0 (S304). In a case where the remainder does not become 0 (S304: No), the information processing apparatus 300 returns to the operation of step S302 and records the sensing values of each of the sensors S₁ to S_{N} at the next time in the memory, or the like.

On the other hand, in a case where the remainder becomes 0 (S304: Yes), the information processing apparatus 300 calculates cross-correlation functions between each of the sensors and other sensors in the cross-correlation calculation unit 301 (S305). Next, the information processing apparatus 300 causes the excluded sensor setting unit 302 to set a sensor in which the maximum values of the absolute values of the cross-correlation functions are all less than the threshold as a sensor not to be used for deriving the pulse wave propagation speed (S306). In this event, the information processing apparatus 300 may set a sensor that is not to be used for deriving the pulse wave propagation speed using the normalized cross-correlation functions instead of the normal cross-correlation functions.

Subsequently, in the information processing apparatus 300, the pulse wave propagation period derivation unit 303 derives the pulse wave propagation period from the deviation of the maximum value of the cross-correlation function in each of the combinations of the sensors excluding the sensor set not to be used (S307). Thereafter, in the information processing apparatus 300, the pulse wave propagation speed derivation unit 304 acquires distance information between the sensors (S308), and derives the pulse wave propagation speed in each combination of the sensors (S309). Furthermore, the information processing apparatus 300 derives the pulse wave propagation speed as a whole by averaging the pulse wave propagation speeds in the derived combinations of the sensors (S310).

This enables the information processing apparatus 300 to derive the pulse wave propagation speed every Q seconds. Thereafter, the information processing apparatus 300 returns to the operation of step S302 and records each of the sensing values of the sensors S₁ to S_{N} at the next time in the memory, or the like.

According to the above operation, the information processing apparatus 300 can derive the pulse wave propagation speed every Q seconds by selectively using the pulse wave signals of the sensors that favorably sense the pulse wave. Furthermore, the information processing apparatus 300 can derive the pulse wave propagation speed with higher accuracy by further averaging the pulse wave propagation speeds derived from the pulse wave signals of the sensors that favorably sense the pulse wave.

### 3.3. Modification

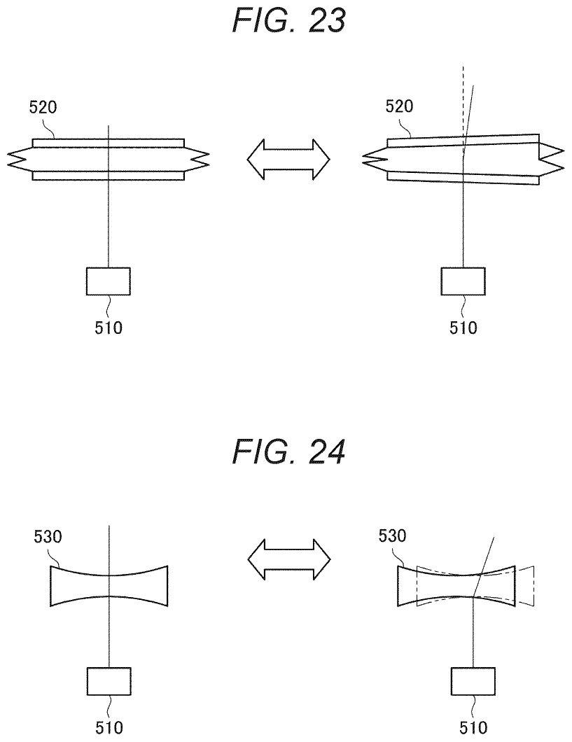

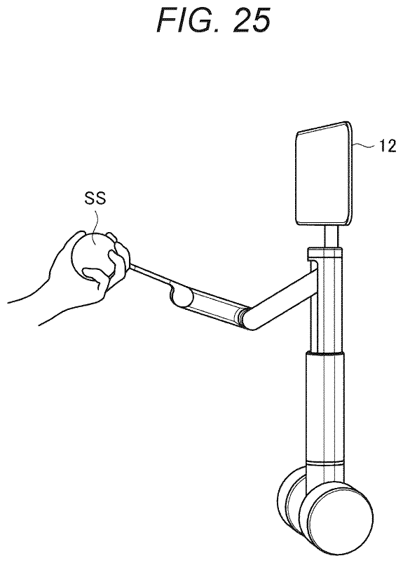

A modification of the information processing apparatus 300 according to the present embodiment will be described with reference to FIGS. 23 and 24. In the modification according to the present embodiment, the information processing apparatus 300 can execute operation of improving signal quality from the sensor having a low cross-correlation. FIGS. 23 and 24 are schematic views illustrating a correction mechanism that corrects a sensing position of light emitted from a sensor in order to improve signal quality from the sensor.

Specifically, the information processing apparatus 300 may execute operation of improving the signal quality in order to enhance the cross-correlations with other sensors for the sensor in which the maximum values of the absolute values of the cross-correlation functions with other sensors are all less than the threshold.

As an example, the information processing apparatus 300 may perform processing such as noise removal on the pulse wave signal sensed by the sensor in which the maximum values of the absolute values of the cross-correlation functions with other sensors are all less than the threshold. The information processing apparatus 300 can improve signal quality by reducing noise from a pulse wave signal with low signal quality.

As another example, the information processing apparatus 300 may perform operation of correcting a pulse wave sensing position of the sensor in which the maximum values of the absolute values of the cross-correlation functions with other sensors are all less than the threshold. For example, the information processing apparatus 300 may correct the sensing position so that the light emitted from the sensor can more easily sense the artery by operating the correction mechanism that corrects the pulse wave sensing position.

As illustrated in FIG. 23, the information processing apparatus 300 may correct an irradiation position of light from a light source 510 of the sensor by using a vari-angle prism 520 in which a space between two sheets of glass plates and a bellows-shaped tube is filled with a liquid having a high refractive index. As illustrated in FIG. 24, the information processing apparatus 300 may correct the irradiation position of the light from the light source 510 of the sensor by using a correction optical system 530 including a concave lens, and the like.

In the modification according to the present embodiment, the information processing apparatus 300 can efficiently improve accuracy of the derived pulse wave propagation speed by performing the operation of improving the signal quality only for the sensor having a low cross-correlation and considered not to favorably sense the pulse wave.

### 3.4. Example of Measurement Device

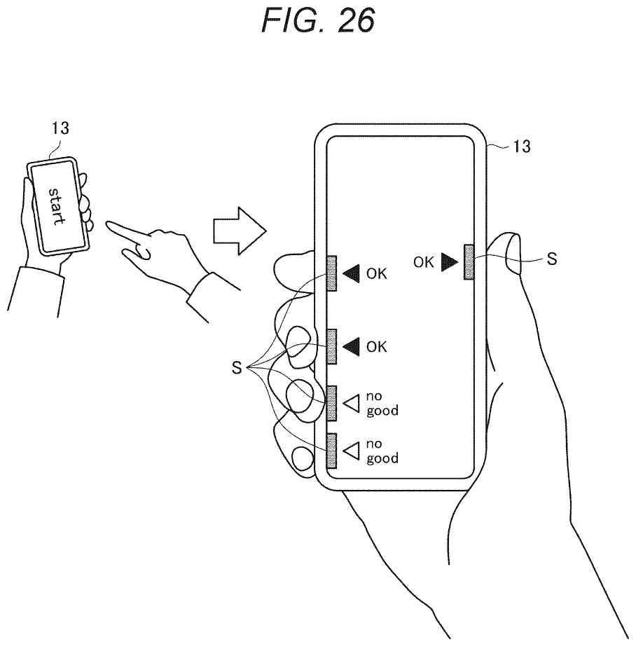

Next, with reference to FIGS. 25 to 28, first to fourth examples of the measurement device provided with the sensors S₁ to S_{N} (hereinafter, also collectively referred to as the sensor S) in the present embodiment will be described.

### First Example

FIG. 25 is a schematic view illustrating an appearance of a measurement device 12 according to the first example.

As illustrated in FIG. 25, the measurement device 12 according to the first example may be a robot device including a movement mechanism that autonomously moves the measurement device 1, a display device that displays an instruction, or the like, to the subject US for whom the blood pressure is to be measured, and the measurement unit SS including the sensor that senses a pulse wave.

The measurement unit SS is a spherical body in which a plurality of sensors for sensing a pulse wave is arranged on a surface. The measurement unit SS can sense the pulse wave of the subject US with a plurality of sensors arranged on the surface by being held by the hand of the subject US.

This enables the measurement device 12 to measure the pulse wave propagation speed and the blood pressure of the subject US using the outputs from the sensors that favorably sense the pulse wave regardless of how the subject US grips the measurement unit SS. In addition, the measurement device 12 can move autonomously by the movement mechanism, so that the measurement device 12 can move near the subject US and measure the blood pressure of the subject US when it is necessary to measure the blood pressure.

### Second Example

FIG. 26 is a schematic view illustrating an appearance of a measurement device 13 according to a second example.

As illustrated in FIG. 26, the measurement device 13 according to the second example may be a terminal device possessed by the subject US for whom the blood pressure is to be measured. The terminal device functioning as the measurement device 13 may be a smartphone, a tablet terminal, a laptop computer, or the like. Specifically, the sensor S of the measurement device 13 is disposed on each side surface of the terminal device. The measurement device 13 may instruct the subject US to bring the body surface into contact with the sensor S using image display, or the like, and may indicate to the subject US whether or not the contact of the body surface with the sensor S is favorable.

This enables the measurement device 13 to give an instruction of a gripping state of the measurement device 13 by the subject US using the image display, so that it is possible to measure the pulse wave propagation speed and the blood pressure of the subject US with higher accuracy. In addition, the measurement device 13 is configured as a terminal device such as a smartphone, a tablet terminal, or a laptop computer, so that it is possible to cause the subject US to more easily measure the blood pressure.

### Third Example

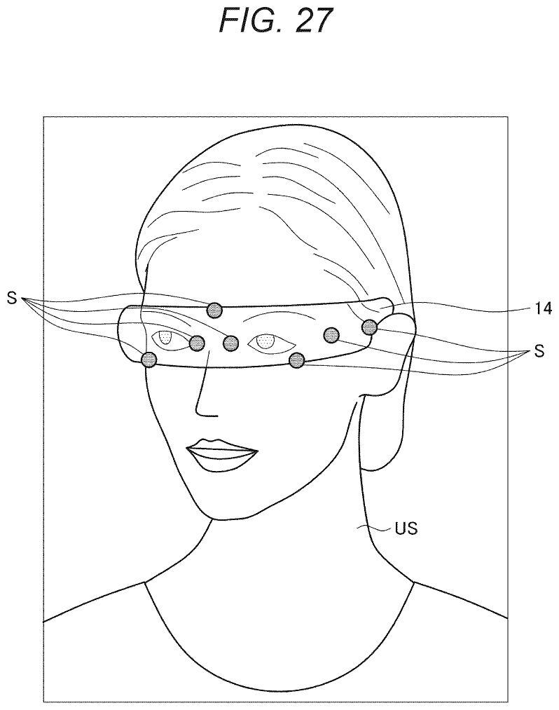

FIG. 27 is a schematic view illustrating an appearance of a measurement device 14 according to a third example.

As illustrated in FIG. 27, the measurement device 14 according to the third example may be smart glasses or goggles to be worn by the subject US for whom the blood pressure is to be measured. Specifically, the sensor S of the measurement device 14 may be provided on a bridge, a nose pad, a rim, a temple, a modern, or the like, which is an approach point between the smart glasses or the goggles and the face of the subject US.

This enables the measurement device 14 to measure the pulse wave propagation speed and the blood pressure of the subject US with higher accuracy using the pulse wave signals sensed at a plurality of positions on the face of the subject US. In addition, the measurement device 14 can monitor the blood pressure of the subject US who is using the smart glasses or the goggles at an arbitrary timing.

### Fourth Example

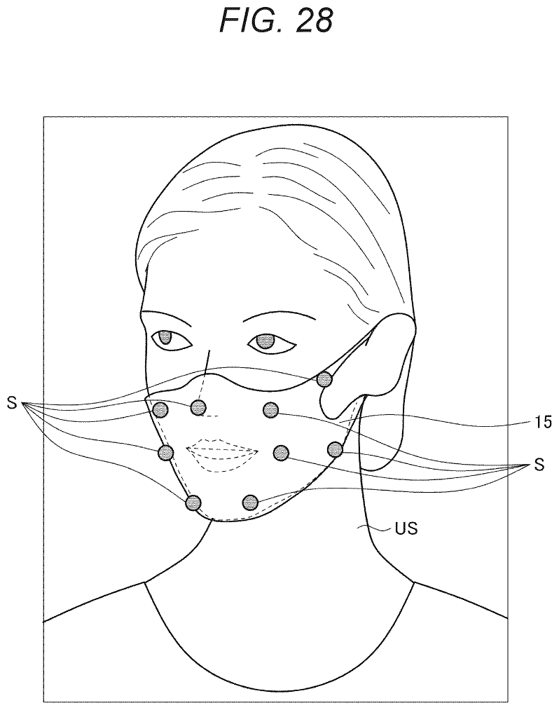

FIG. 28 is a schematic view illustrating an appearance of a measurement device 15 according to a fourth example.

As illustrated in FIG. 28, the measurement device 15 according to the fourth example may be a mask to be worn by the subject US for whom the blood pressure is to be measured. Specifically, the sensor S of the measurement device 15 may be provided at an approach point to the face of the subject US on the back side of the mask.

This enables the measurement device 15 to measure the pulse wave propagation speed and the blood pressure of the subject US with higher accuracy using the pulse wave signals sensed at a plurality of positions on the face of the subject US. In addition, the measurement device 15 can monitor the blood pressure of the subject US who is using the mask at an arbitrary timing.

Although the preferred embodiments of the present disclosure have been described above in detail with reference to the accompanying drawings, the technical scope of the present disclosure is not limited to such an example. It is obvious that those with ordinary skill in the technical field of the present disclosure can conceive various alterations or corrections within the scope of the technical idea recited in the claims, and it is naturally understood that these alterations or corrections also fall within the technical scope of the present disclosure.

Note that the functions of the information processing apparatus according to each embodiment of the present disclosure can be implemented by cooperation of software and hardware. The information processing apparatus according to each embodiment of the present disclosure may include hardware such as a central processing unit (CPU), a read only memory (ROM), and a random access memory (RAM).

For example, the functions of the fitting unit 101, the peak position estimation unit 102, the averaging unit 201, the peak detection unit 202, the cross-correlation calculation unit 301, the excluded sensor setting unit 302, the pulse wave propagation period derivation unit 303, the pulse wave propagation speed derivation units 103, 203, 304, and the blood pressure derivation unit 400 may be executed by, for example, a CPU.

In addition, it is also possible to create a program for causing hardware such as a CPU, a ROM, and a RAM built in a computer to exhibit functions equivalent to those of the above-described information processing apparatus. Furthermore, a computer-readable recording medium in which the program is recorded can also be provided.

The effects described in the present specification are merely illustrative or exemplary and are not restrictive. In other words, the technology according to the present disclosure can exhibit other effects apparent to those skilled in the art from the description of the present specification, in addition to the effects described above or instead of the effects described above.

Note that the following configurations also fall within the technological scope of the present disclosure.
(1)
An information processing apparatus including: a calculation unit
configured to derive a pulse wave propagation speed of a subject by performing signal processing together on pulse wave signals sensed at three or more different positions on a body surface of the subject.
(2)
The information processing apparatus according to (1), in which the calculation unit estimates peak positions of the pulse wave signals by fitting each of the pulse wave signals sensed at the same time at different positions on the same arterial path to a predetermined pulse waveform, and derives the pulse wave propagation speed on the basis of a difference between the peak positions of the pulse wave signals at different times.
(3)
The information processing apparatus according to (2), in which the peak positions of the pulse wave signals are estimated at a first interval, and
the pulse wave propagation speed is derived at a second interval longer than the first interval.
(4)
The information processing apparatus according to (2) or (3), in which the predetermined pulse waveform is set on the basis of the pulse wave signals of the subject.
(5)
The information processing apparatus according to (1), in which the calculation unit derives a peak of an average pulse wave signal obtained by averaging each of the pulse wave signals sensed in the same time section at substantially the same distance from the heart, and derives the pulse wave propagation speed on the basis of the peak of the average pulse wave signal and an electrocardiogram peak of the subject.
(6)

The information processing apparatus according to (1), in which the calculation unit derives the pulse wave propagation speed using the pulse wave signals in which cross-correlations between the pulse wave signals are equal to or higher than a threshold among each of the pulse wave signals sensed in the same time section at an arbitrary position on the same arterial path.
(7)
The information processing apparatus according to (6), in which the calculation unit derives the pulse wave propagation speed as a whole by averaging individual pulse wave propagation speeds derived from each of combinations of the pulse wave signals in which the cross-correlations between the pulse wave signals are equal to or higher than the threshold.
(8)
The information processing apparatus according to (7), in which the calculation unit averages the individual pulse wave propagation speeds by using weighting based on lengths of distances between positions where the pulse wave signals are sensed.
(9)
The information processing apparatus according to any one of (6) to (8), in which the calculation unit derives the pulse wave propagation speed in further consideration of gravity acting on the body surface of the subject.
(10)
The information processing apparatus according to any one of (1) to (9), in which the pulse wave signals are sensed by an optical sensor.
(11)
The information processing apparatus according to any one of (1) to (10), further including a blood pressure derivation unit configured to derive a blood pressure of the subject on the basis of the pulse wave propagation speed.
(12)
The information processing apparatus according to (11), in which the blood pressure derived by the blood pressure derivation unit is calibrated using a reference blood pressure measured by a sphygmomanometer.
(13)
The information processing apparatus according to any one of (1) to (12), in which the body surface of the subject for whom pulse wave signals are to be sensed is a surface of a hand, a foot, or a head.
(14)
An information processing method including:
deriving, by an arithmetic processing device, a pulse wave propagation speed of a subject by performing signal processing together on pulse wave signals sensed at three or more different positions on a body surface of the subject.
(15)
A program for causing a computer to function as a calculation unit configured to derive a pulse wave propagation speed of a subject by performing signal processing together on pulse wave signals sensed at three or more different positions on a body surface of the subject.

### REFERENCE SIGNS LIST

100, 200, 300 Information processing apparatus
101 Fitting unit
102 Peak position estimation unit
103 Pulse wave propagation speed derivation unit
121 Sensor information storage unit
122 Pulse waveform storage unit
201 Averaging unit
202 Peak detection unit
203 Pulse wave propagation speed derivation unit
301 Cross-correlation calculation unit
302 Excluded sensor setting unit
303 Pulse wave propagation period derivation unit
304 Pulse wave propagation speed derivation unit
321 Sensor information storage unit
400 Blood pressure derivation unit
S Sensor
SS Measurement unit
US Subject

1. An information processing apparatus comprising:
a calculation unit configured to derive a pulse wave propagation speed of a subject by performing signal processing together on pulse wave signals sensed at three or more different positions on a body surface of the subject.

2. The information processing apparatus according to claim 1, wherein the calculation unit estimates peak positions of the pulse wave signals by fitting each of the pulse wave signals sensed at a same time at different positions on a same arterial path to a predetermined pulse waveform, and derives the pulse wave propagation speed on a basis of a difference between the peak positions of the pulse wave signals at different times.

3. The information processing apparatus according to claim 2, wherein the peak positions of the pulse wave signals are estimated at a first interval, and
the pulse wave propagation speed is derived at a second interval longer than the first interval.

4. The information processing apparatus according to claim 2, wherein the predetermined pulse waveform is set on a basis of the pulse wave signals of the subject.

5. The information processing apparatus according to claim 1, wherein the calculation unit derives a peak of an average pulse wave signal obtained by averaging each of the pulse wave signals sensed in a same time section at substantially the same distance from the heart, and derives the pulse wave propagation speed on a basis of the peak of the average pulse wave signal and an electrocardiogram peak of the subject.

6. The information processing apparatus according to claim 1, wherein the calculation unit derives the pulse wave propagation speed using the pulse wave signals in which cross-correlations between the pulse wave signals are equal to or higher than a threshold among each of the pulse wave signals sensed in a same time section at an arbitrary position on a same arterial path.

7. The information processing apparatus according to claim 6, wherein the calculation unit derives the pulse wave propagation speed as a whole by averaging individual pulse wave propagation speeds derived from each of combinations of the pulse wave signals in which the cross-correlations between the pulse wave signals are equal to or higher than the threshold.

8. The information processing apparatus according to claim 7, wherein the calculation unit averages the individual pulse wave propagation speeds by using weighting based on lengths of distances between positions where the pulse wave signals are sensed.

9. The information processing apparatus according to claim 6, wherein the calculation unit derives the pulse wave propagation speed in further consideration of gravity acting on the body surface of the subject.

10. The information processing apparatus according to claim 1, wherein the pulse wave signals are sensed by an optical sensor.

11. The information processing apparatus according to claim 1, further comprising a blood pressure derivation unit configured to derive a blood pressure of the subject on a basis of the pulse wave propagation speed.

12. The information processing apparatus according to claim 11, wherein the blood pressure derived by the blood pressure derivation unit is calibrated using a reference blood pressure measured by a sphygmomanometer.

13. The information processing apparatus according to claim 1, wherein the body surface of the subject for whom the pulse wave signals are to be sensed is a surface of a hand, a foot, or a head.

14. An information processing method comprising:
deriving, by an arithmetic processing device,
a pulse wave propagation speed of a subject by performing signal processing together on pulse wave signals sensed at three or more different positions on a body surface of the subject.

15. A program causing a computer to function as:
a calculation unit configured to derive a pulse wave propagation speed of a subject by performing signal processing together on pulse wave signals sensed at three or more different positions on a body surface of the subject.
